(19) United States
(12) Patent Application Publication
(10) Pub. No.: US 2022/0054061 A1
(43) Pub. Date: Feb. 24, 2022

(54) PORTABLE PHLEBOTOMY STATION WITH INTERNAL STORAGE

(71) Applicant: Icahn School of Medicine at Mount Sinai, New York, NY (US)

(72) Inventor: Hennesys Disla, Wayne, NJ (US)

(21) Appl. No.: 17/335,320

(22) Filed: Jun. 1, 2021

Related U.S. Application Data
(63) Continuation of application No. 15/947,617, filed on Apr. 6, 2018, now abandoned.
(60) Provisional application No. 62/482,452, filed on Apr. 6, 2017.

(51) Int. Cl. A61B 5/15; A61B 50/18; A61B 50/31

(52) U.S. Cl. CPC A61B 5/150206; A61B 50/18; A61B 50/312; A61B 2050/311; A61B 2050/185; A61B 5/15003; A61B 5/150748; A61B 50/31

(57) ABSTRACT

A portable medical storage unit (station) is configured in the form of a phlebotomy arm wedge that includes internal storage along with other optional features to facilitate a health professional to perform a phlebotomy. The portable medical storage unit thus provides an alternative product to a phlebotomy chair and has a much smaller footprint and can easily be stored in closet or the like. Moreover, the portable medical storage unit can be made at much less cost than a phlebotomy chair.

### CROSS-REFERENCE TO RELATED PATENT APPLICATIONS

This application is a continuation of U.S. patent application Ser. No. 15/947,617, filed Apr. 6, 2018, which is based on and claims priority to U.S. Provisional Patent Application 62/482,452, filed Apr. 6, 2017, the entire contents of which is incorporated by reference herein as if expressly set forth in its respective entirety herein.

### TECHNICAL FIELD

The present invention is directed to a portable phlebotomy station (unit or kit) that has internal storage to allow storage of certain medical supplies and more particularly, relates to a portable phlebotomy station that is shaped like a wedge so as to define an integral armrest along a top surface thereof that is intended for use while performing a phlebotomy procedure and further includes internal storage for medical supplies.

### BACKGROUND

As is known, medical procedures and treatments not only often require specific equipment but also require medical supplies that are used to perform a certain medical procedure, etc. Even the simplest treatment, such as treatment of a scrape on the body, involves the use of a number of medical supplies, such as gloves to be worn by the person treating the scrape, an antiseptic/cleaning agent, antibiotic ointment, and dressing for the wound.

One common medical procedure that is performed in a number of different settings is the drawing of blood for testing and analysis or even for blood collection efforts. A blood drawing chair, also known as a phlebotomy chair, is a specialized medical room chair for a patient to comfortably sit on to allow medical personnel clear and easy access of the patient to take a blood sample or to draw blood for testing or even to draw blood for blood drive collections. While phlebotomy chairs are used in a number of medical facilities, such as hospitals, physician's offices, nursing homes, and clinics, many clinics and patient households do not have phlebotomy chairs. Several reasons of why they are not used in more location are that these types of chairs are big and can be expensive.

There is therefore a desired need for an alternative product to the phlebotomy chair which is configured to not only permit the drawing of blood but also provides storage for related medical supplies that are needed to perform this task.

### SUMMARY

One exemplary portable medical storage unit (station) that is disclosed herein is in the form of a phlebotomy arm wedge that includes internal storage along with other optional features to facilitate a health professional to perform a phlebotomy. The portable medical storage unit thus provides an alternative product to a phlebotomy chair and has a much smaller footprint and can easily be stored in closet or the like. Moreover, the portable medical storage unit can be made at much less cost than a phlebotomy chair.

### BRIEF DESCRIPTION OF THE DRAWING FIGURES

FIG. 1 is top and side perspective view of a portable medical station with integral phlebotomy armrest according to one embodiment;

FIG. 2 is a top and side perspective view of the portable medical station in a vertical orientation;

FIG. 3 is a top and side perspective view of the portable medical station with a lid being shown in an open position;

FIG. 4 is a top and side perspective view showing a phlebotomy procedure being performed;

FIG. 5 shows the portable medical station with a carry-on strap;

FIG. 6 shows the cover (lid) in an open position with a latch for a writing implement;

FIGS. 7A-7E show the portable medical station with additional add-on features including a comfort padding being placed along the upper surface of the lid and an outer receptacle according to one or more embodiments;

FIGS. 8A-8B show the portable medical station having a paper dispenser incorporated into the rear of the unit according to at least one embodiment;

FIGS. 9A-9B show an exemplary drawer of the portable medical station, the drawer having a knock-down form in which the drawer is formed of collapsible panels that move between a collapsed (storage) state and an assembled state according to at least one embodiment;

FIGS. 10A-10C show an exemplary stack of disposal sheets bound together, which can be provided and disposed on the top surface of the portable medical station according to at least one embodiment;

FIGS. 11A-11C show an embodiment of the portable medical station in which the portable medical station has a pyramid shape; and

FIGS. 12A-12E show another embodiment of the portable medical station in which the portable medical station has a hemispherical shape.

### DETAILED DESCRIPTION OF CERTAIN EMBODIMENTS

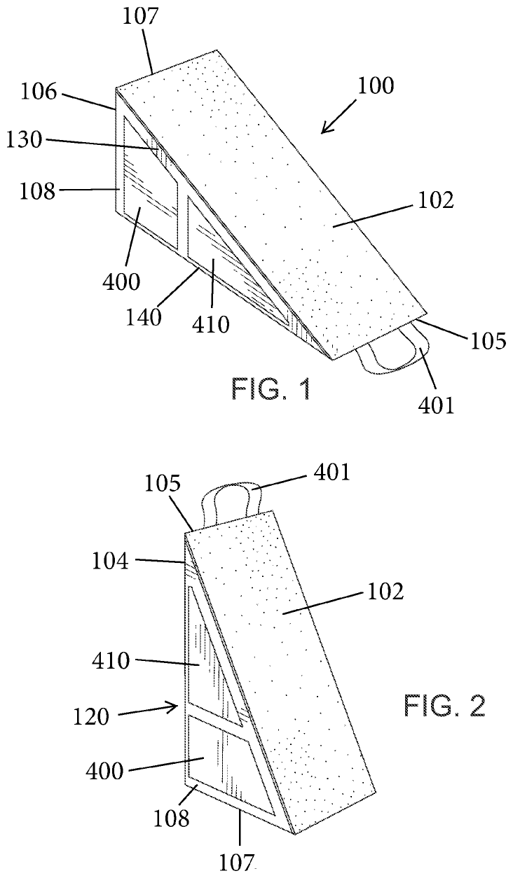

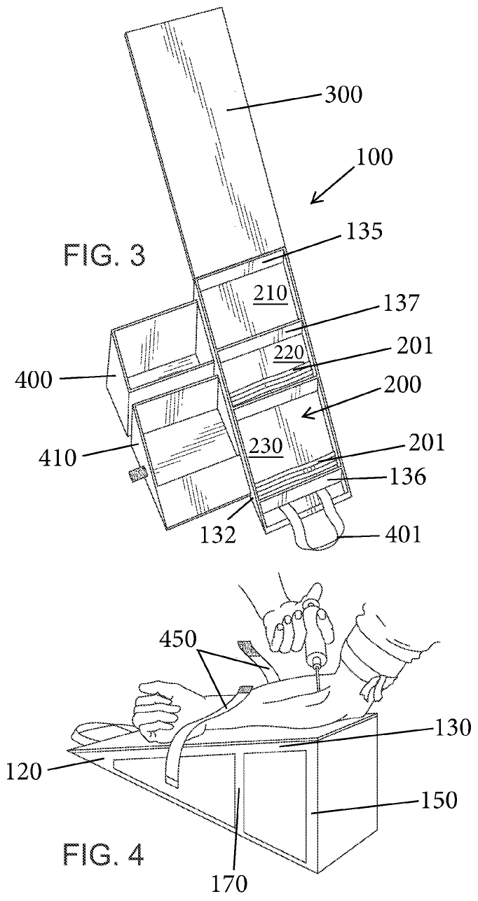

FIGS. 1-4 disclose a portable medical storage unit (station) 100 and more specifically, the portable medical storage unit 100 comprises a phlebotomy arm wedge that includes internal storage along with other optional features to facilitate a health professional to perform a phlebotomy. The unit 100 is portable in that it is configured to be easily carried by a person and is sized so as to be used with human patients.

The unit 100 is thus also referred to herein as a portable phlebotomy unit 100. As is known, a typical procedure for drawing blood involves the following steps: (1) assemble equipment; (2) identify and prepare the patient; (3) select the site; (4) perform hand hygiene and put on gloves; (5) disinfect the entry site; (6) take blood; (7) fill the laboratory sample tubes; (8) draw samples in the correct order; (9) clean contaminated surfaces and complete patient procedure; (10) prepare samples for transportation; and (11) clean up any spills or blood or bodily fluids.

To properly draw blood, the patient's arm needs to be placed on a flat support surface to allow the health professional to properly draw blood using the above procedure. As mentioned above, when a medical clinic or a household does not have a phlebotomy chair, the portable phlebotomy unit 100 provides a suitable alternative to a phlebotomy chair.

As shown in the figures, the portable phlebotomy unit 100 has a wedge-shaped construction and thus is generally triangular shaped. The portable phlebotomy unit 100 has a top surface 102, an opposing base (bottom) surface 104, an end surface 106, a first side 108, and an opposing second side 109. These three surfaces 102, 104, 106 are arranged so as to define a triangle. In other words, the top surface 102 and bottom surface 104 converge toward one another and intersect one another at a front edge 105 of the unit 100. The top and bottom surfaces 102, 104 diverge from one another at a rear edge 107 of the unit 100. The end surface 106 intersects both the top and bottom surfaces 102, 104, with the end surface 106 being formed perpendicular to the bottom surface 104 (an acute angle is formed between the top surface 102 and the end surface 106).

The portable phlebotomy unit 100 is defined by a body (frame) 120 that includes a first wall 130, an opposing second wall 140 that is formed at an angle relative to the first wall 130, and an end wall 150 that extends between the first wall 130 and the second wall 140. The first wall 130 and the second wall 140 intersect at the front edge 105 and a right angle is formed between the second wall 140 and the end wall 150. It will be understood that the walls, 130, 140, 150 can be an integral structure and formed as a single structure.

A hollow interior space is formed between the walls 130, 140, 150. Within the hollow interior space, one or more divider walls 170 can be provided (the divider wall 170 can be formed as a single integral structure with walls 130, 140, 150. Each divider wall 170 extends between the first wall 130 and the second wall 140 and serves to divide the hollow interior space into one or more compartments. In the illustrated embodiment, there is a single divider wall 170 that partitions the hollow interior space into a first compartment and an adjacent second compartment. As illustrated, the first compartment has a trapezoidal shape, while the second compartment has a triangular shape.

In the illustrated embodiment, both the first and second compartments are open along both the first side 108 and the opposing second side 109. It will also be understood that the location of the divider wall 170 determines the relative sizes of the first and second compartments.

The first wall 130 has a first upstanding rail 132 that extends upwardly therefrom along the first side 108 and an opposing second upstanding rail 134 that extends upwardly therefrom along the second side 109. At one end of the first and second upstanding rails 132, 134, there is a rear upstanding rail 135 that extends between the first and second upstanding rails 132, 134 and an opposite end, a front upstanding rail 136 extends between the first and second upstanding rails 132, 134. In addition, one or more divider rails 137 can be provided between the first and second upstanding rails 132, 134. An upper storage space 200 is defined between the rails 132, 134, 135, 136 and each divider rail 137 partitions the upper storage space 200 into compartments. In the illustrated embodiment, there are two divider rails 137 that partition the upper storage space 200 into a first upper storage compartment 210, a second upper storage compartment 220, and a third upper storage compartment 230. It will be appreciated that the first wall 130 represents a floor of the upper storage space 200 and the rails 132, 134, 135, 136 define side and end walls of the upper storage space 200.

Each of the upper storage compartments 210, 220, 230 is configured to receive and store certain articles, in this case medical supplies. One or more of the upper storage compartments 210, 220, 230 can include one or more elastic straps 201 that are configured to hold medical supplies in place in the respective compartment by lifting the elastic strap 201 and placing the medical supply thereunder.

A pivotable cover or lid 300 is pivotally attached to the body 120 along the rear thereof. In particular, the pivotable lid 300 can pivot about the rear rail 135 and is configured to seat against upper edges of the first and second upstanding rails 132, 134 and the front upstanding rail 136 so as to close off each of the upper storage compartments 210, 220, 230. An outer surface of the pivotable lid 300 defines the top surface 102 of the unit 100.

It will be understood that the top surface 102 is preferably a smooth, flat surface on which the patient's arm is placed. The top surface 102 can include a layer of material to assist in the phlebotomy and in particular, the layer of material can be an anti-skid material or a comfort material. In an in-use position, which is shown in FIG. 1, the unit 100 is positioned such that the bottom (second) wall 140 is placed on a support structure, which can be in the form of a table, counter or even the leg of a sitting patient. In this in-use position, the top (first) wall 130 and the pivotable lid 300 face upright.

It will also be understood that the pivotable lid 300 can have a lock or latch feature to ensure that the pivotable lid 300 remains closed in a covering relationship over the upper storage space 200. For example, the pivotable lid 300 can have a first fastener that mates with a second fastener that is part of the body 120. In one embodiment, the first fastener can be a hook and loop tab at the front edge of the lid 300 that mates with hook and loop material that is coupled to the body 120. Other types of fasteners, such as snaps, buttons, etc., can be used. In addition, a mechanical lock device can likewise be used to lock the pivotable cover 300 in place over the body 120.

The portable phlebotomy unit 100 also includes one or more drawers and in the illustrated embodiment, the unit 100 includes a first drawer 400 and a second drawer 410. The first drawer 400 is configured to be slidingly received within the first compartment 180 and the second drawer 410 is configured to be slidingly received within the second compartment 190. The first drawer 400 thus has a trapezoidal shape and the second drawer 410 has a triangular shape.

Each of the drawers 400, 410 has a base (floor) and opposing side walls and end walls that defines a drawer storage space for receiving and storing certain articles, in this case medical supplies. In the event that the first and second compartments 180, 190 are open along both the first side 108 and the second side 109, the drawers 400, 410 can be inserted into and pulled outward from either the first side 108 and second side 109. This allows both left handed and right-handed health personnel to prepare the patient and withdraw blood since access to supplies in the drawers 400, 410 can be accessed either along the first side 108 or the second side 109. The drawers 400, 410 thus slide along the second (bottom) wall 140.

It will be understood that the drawers 400, 410 can be securely held in place and/or locked using any number of different conventional techniques. For example, the hook and loop material (e.g., straps/tabs) can be used to secure the drawers in place and/or a locking system can be used to releasably lock the drawers 400, 410 in place.

The upper storage space 200 and the drawers 400, 410 can hold medical supplies that are typically used in a phlebotomy procedure. Such supplies can include but are not limited to: (1) a supply of laboratory sample tubes; (2) sterile glass or plastic tubes with rubber caps; (3) well-fitting gloves; (4) an assortment of blood-sampling devices (safety-engineered devices or needles and syringes); (5) a tourniquet; (6) alcohol hand rub; (7) alcohol swabs (prep pads) for skin disinfection; (8) gauze or cotton-wool ball to be applied over puncture site; (9) bandages; (10) laboratory specimen labels; (11) writing implement; (12) laboratory forms; (13) leak-proof transportation bags or containers, etc.

A handle 401 can be attached to the front upright rail 136 and is configured such that the handle is exposed and accessible when the pivotable lid 300 is closed. The handle 401 can be a strap (loop) that can easily be grasped by a person to move and/or carry the unit 100 from one place to another. As shown in FIG. 2, the unit 100 can also be stored in a vertical manner by being placed on its end wall. In addition, two or more units 100 can be stacked in a compact manner by placing a top unit 100 with its rear edge superimposed over a front edge of the bottom unit 100. Combines the two units 100 will have a rectangular shape.

It will also be appreciated that the underside of the bottom wall 140 can include a non-slip grip material (layer) for placement on the patient's lap, table or bed allowing a straight needle to puncture the skin at the desired location.

In addition, the unit 100 a pair of straps 450 that can be used to securely locate and hold the patient's arm on the arm rest component of the unit 100 (top surface 102). The straps 450 can be attached to the body 120 of the unit 100 and free ends of the strap have complementary fasteners, such as hook and loop material, buttons, snaps, etc. One strap 450 extends from the first side 108, while the other strap 450 extends from the second side 109 and can be placed over and joined together over the patient's arm. The straps 450 can alternatively be attached to one of the drawers 400, 410 as by extending along an underside of the drawer 400, 410. Alternatively, the straps 450 can extend through a slit formed along the sides of the drawer 400, 410.

The unit 100 is an effective alternative to a traditional phlebotomy chair and is useful for emergency medicine, nursing homes (specifically for bed bound patients), or clinics. The unit 100 allows the arm to sit at a better angle for the needle preventing flat need sticks resulting in missed veins (i.e., the vein is easier to view with an elevated arm rest). Conventional portable products fail to deliver the combination of utility (providing arm support for the patient) and storage (to allow the unit to be truly portable).

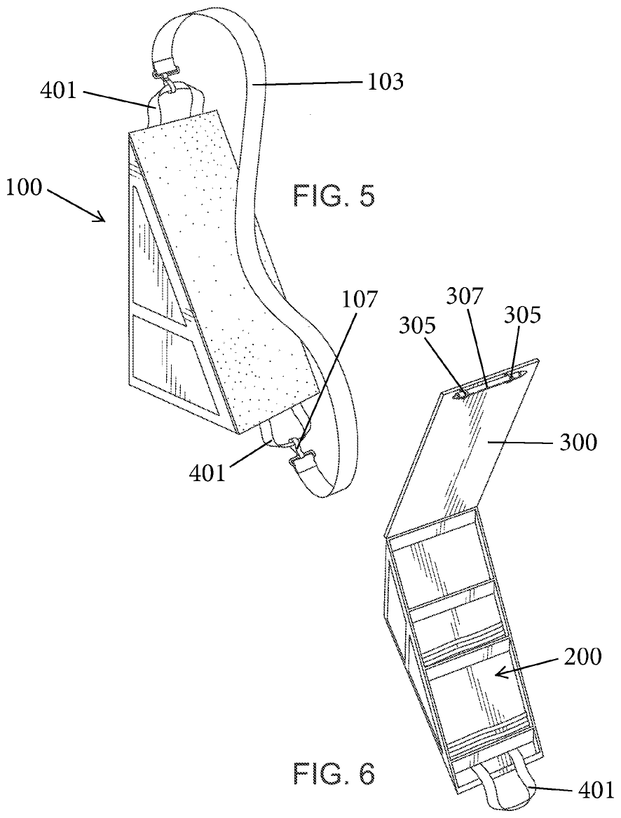

FIG. 5 shows the unit 100 with an optional carry-on strap 103 that is attached at one end of the strap to the one end of the unit 100 and the other end of the strap is attached to the other end of the unit 100. The strap 103 can be attached using traditional techniques including having clips 107 at the ends of the strap 103 that detachably attach to handle straps, such as strap 401. The strap 103 is for placement over a shoulder to allow the unit 100 to be easily transported from one location to another.

FIG. 6 shows that an underside of the pivotable lid 300 can include a holder 305 for receiving and holding a writing implement 307, such as a pen. The holder 305 thus holds the writing implement 307 along the underside. The holder 305 can be in the form of an elastic loop or other structure that can hold the writing implement 307. Alternatively, the holder 305 can be in the form of a latch.

It will also be appreciated that there are a number of additional add-on features that can be incorporated into the unit 100 and moreover, the portable phlebotomy unit can take many different forms as described in further detail below.

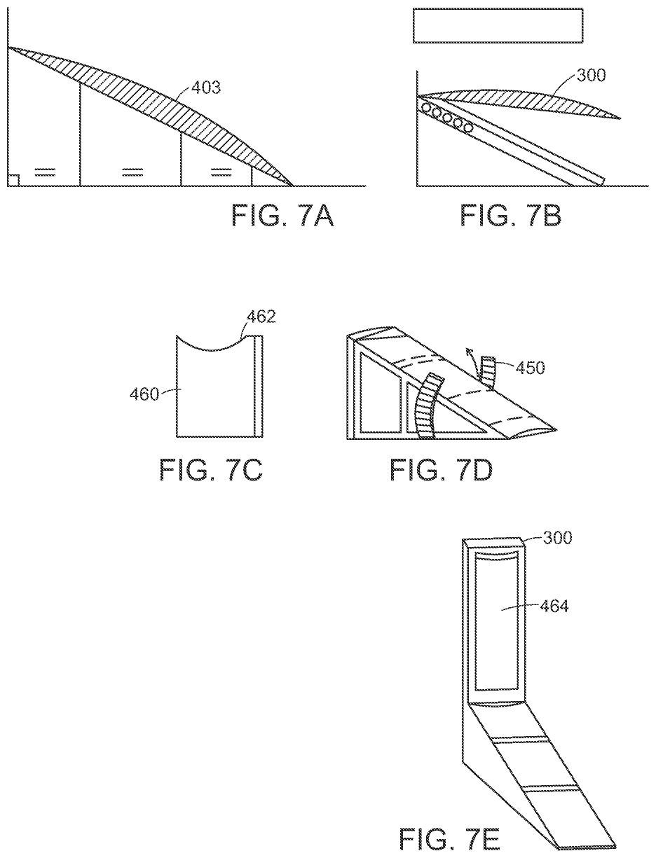

FIGS. 7A-7E show additional add-on features for the unit 100. For example, as shown in FIG. 7C, an outer receptacle 460 can be provided and includes a hollow body in which either a supply of fresh plastic bags can be held or alternatively, the outer receptacle 460 can act as a trash receptacle in which trash can be placed. In the event that the outer receptacle 460 acts as a storage location for fresh plastic bags, then an inner surface of the lid 300 can optionally include a mounted trash bag 464 for discarding waste (see, FIG. 7E). Once the bag is full, a fresh bag from the receptacle 460 is retrieved.

Alternatively, a trash bag is placed into the receptacle 460 for storing trash. An open top end 462 (FIG. 7C) of the trash receptacle 460 can include a pivotable lid or cover. The cover can pivot about a living hinge that is formed as part of the receptacle 460. The receptacle 460 can be releasably coupled to the unit 100 using any number of different techniques, including the use of fasteners or other mechanical coupling techniques. For example, hook and loop material can be used to releasably attach the receptacle 460 to the end wall 150. In addition, other types of fasteners, such as snaps, can be used or a male/female coupling arrangement can be used, such as including protrusions on the receptacle 460 that are received into locking slots formed in the end wall, whereby the receptacle 460 can be effectively suspended (hung) on the end wall 150.

In addition, when the outer receptacle 460 is a trash receptacle, an inner surface of the lid 300 can include one or more trash bags that can be part of a dispenser that is configured for dispensing to the health professional.

FIG. 7A shows a comfort padding 403 being placed along the upper surface of the lid 300 according to at least one embodiment.

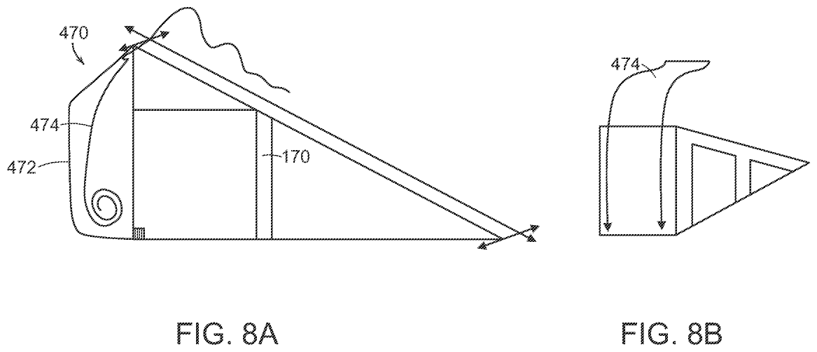

FIGS. 8A-8B shows that a paper dispenser 470 can be incorporated into the rear of the unit 100. As shown in FIG. 8A, the paper dispenser 470 can take the form of a receptacle 472 which can be the same or similar to receptacle 460 (FIG. 7C) but instead stores a roll of paper 474. A lid or cover 474, which can be similar to cover 300, can define a slit or can have a slit formed therein through which the paper 474 travels toward the top surface of the unit. Alternatively, the lid or cover 474 can be eliminated and the paper 474 is simply fed through the open top of the receptacle. The paper 474 is intended to cover the area on which the patient's arm is laid. Once the phlebotomy procedure is completed, the user paper is discarded. The paper 474 can be provided on a roller that permits easy dispensing thereof.

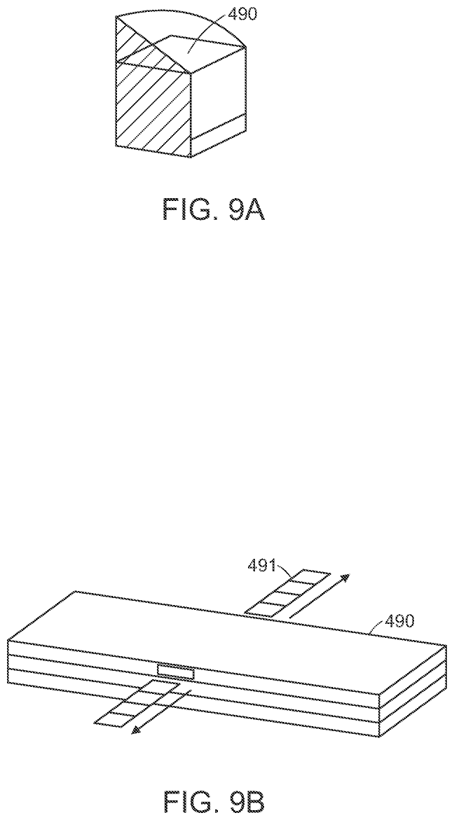

FIGS. 9A-9B show additional features of exemplary portable phlebotomy units according to at least one embodiment. In particular, FIGS. 9A-9B show an exemplary drawer 490 of the portable medical station. As shown in FIGS. 9A-9B, the drawers 490 can have a knock-down form in which the drawer 490 is formed of collapsible panels that move between a collapsed (storage) state (FIG. 9B) and an assembled state (FIG. 9A). FIG. 9A shows hook and look straps 491 and the drawer can be formed of panels and optionally tubes for a frame.

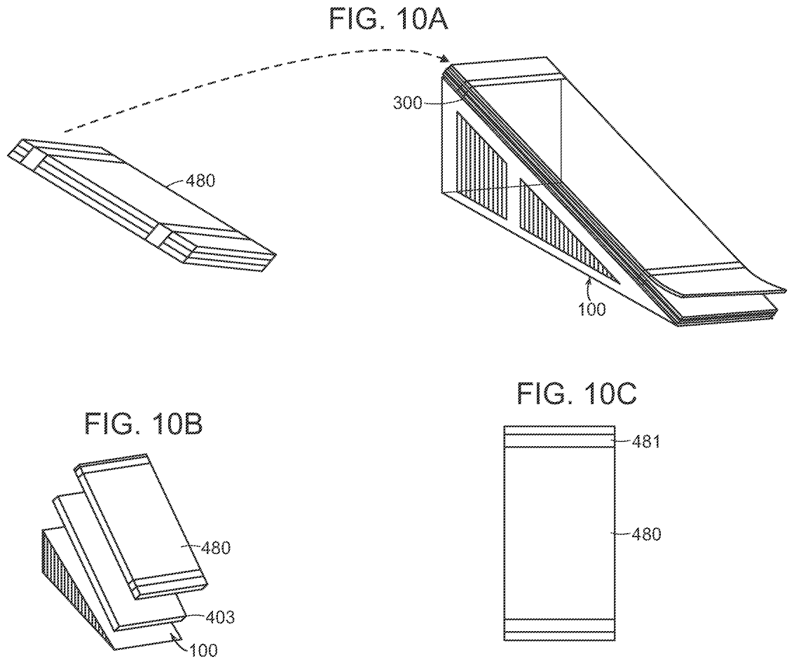

FIGS. 10A-10C show another embodiment in which a bound stack of disposable sheets 480 can be provided and disposed on the top surface of the unit 100. The bound stack of disposable sheets 480 is designed so that the patient's arm is laid on top of the topmost sheet 480 and then after the procedure is complete, this topmost sheet 480 is torn off to expose a new topmost sheet 480 for use by the next patient. The stack 480 can be secured to the lid 300 using any number of techniques, including use of adhesives, bonding agents, or mechanical fasteners, etc. FIG. 10C shows adhesive regions or the like 481 on the back surface of the stack 480.

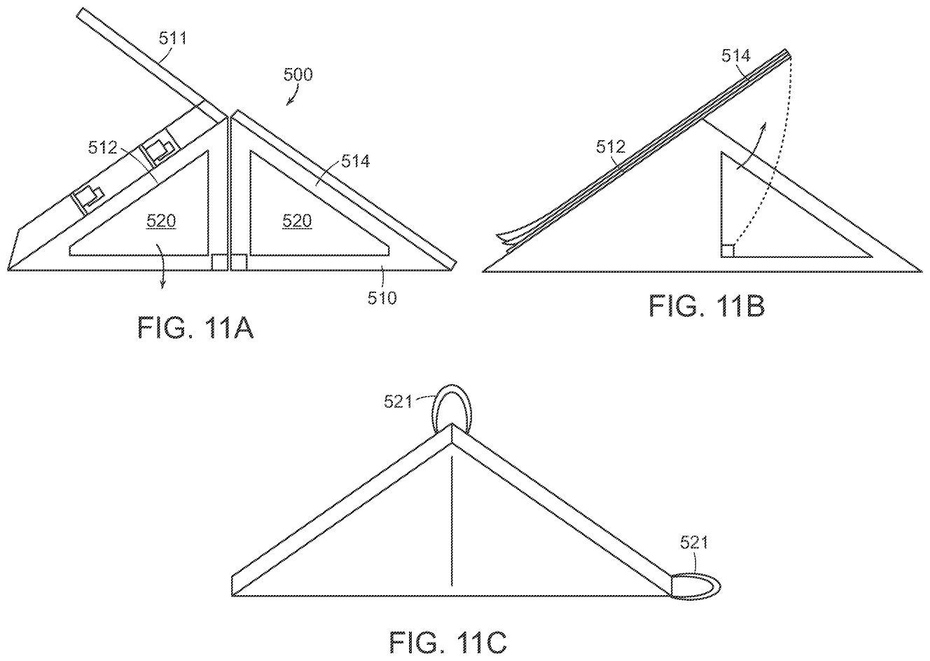

FIGS. 11A-11C illustrate another embodiment of the portable medical station in which the station (unit 500) has a pyramid shape. As shown in FIG. 11A, the unit 500 has a base 510 and first and second top walls 512, 514 that are disposed at an angle to one another such that the unit 500 has a pyramid shape. One or more of the top walls 512, 514 can serve as an openable lid or a lid 511 that pivots open to reveal storage sites can be provided and opens and closes relative to one or more of the top walls 512, 514. In addition, the unit 500, like unit 100, can have one or more drawers 520 that store article and are disposed in a hollow interior of the unit 500 below the top walls 512, 514. One or more handles 521 (FIG. 11C) can be provided. The bound stack of paper sheets 480 (see FIGS. 10A-10B) can be used in this embodiment too.

In addition, as shown in FIG. 11B, in at least one embodiment, the second top wall 514 can be a partial lid that opens and can be configured to pivot into the plane of the other top wall 512 so as to provide an extended length surface on which the arm can be placed. The second top wall 514 thus acts as an extender.

While the unit 500 is shown are containing a pair of triangular shaped drawers 520 (FIG. 11A), it will be appreciated that one of the triangular shaped drawers 520 can comprise two drawers, namely, one smaller triangular shaped drawer and one trapezoidal shape.

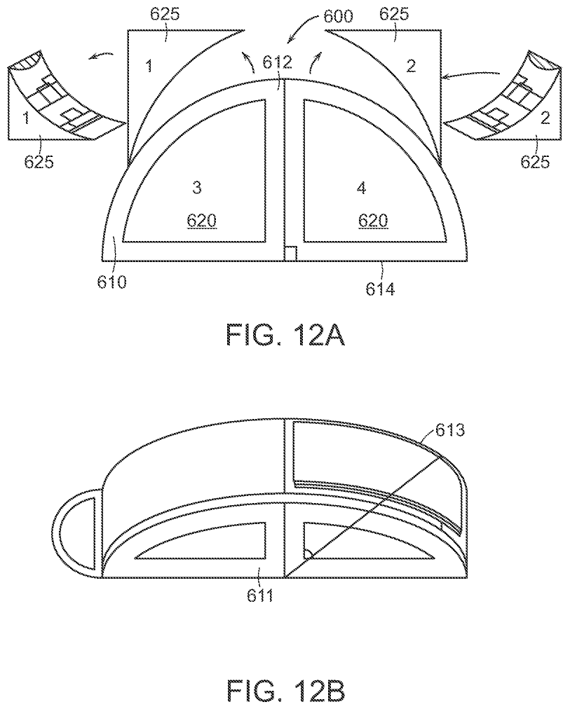

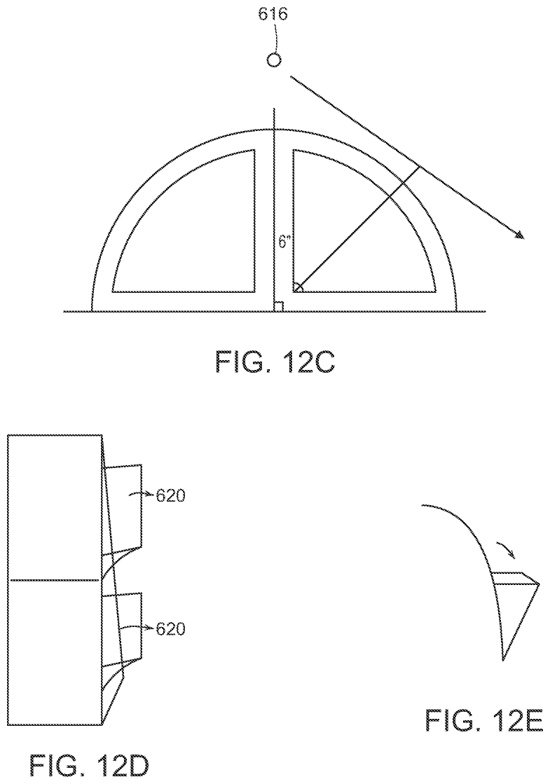

FIGS. 12A-12E illustrate another embodiment of the portable medical station in which the station (unit 600) generally has a hemispherical shape. As shown in FIG. 12A, a body or frame 610 of the unit 600 thus has a curved (convex) shaped top surface 612 and a flat bottom surface 614. As with the other embodiments, the body 610 can be a hollow interior in which one or more drawers 620 are inserted.

In one embodiment, the body/frame 610 is constructed such that the drawers 620 can only be withdrawn along a first side 611 of the unit 600 (see FIGS. 12B and 12D). Along an opposite second side 613 of the unit 600 (see FIG. 12D), the drawers 620 cannot be accessed as for example by having a completely closed side wall along the second side 613. Unlike the other embodiments in which the drawers can be fully removed from the unit, the drawers 620 can be constructed as an attached pivoting drawer in that each drawer is attached along its bottom edge to the body 610. To access the drawer 620, a top edge of the drawer 620 is pulled down causing the drawer to pivot open along its bottom edge (see FIGS. 12D and 12E). The drawer 620 can be attached to the body at its bottom edge. In addition, other drawers or organizing features 625 can be formed and configured to open along the top curved surface 612 as shown in Fig. As shown in FIG. 12B, a handle can be provided.

As shown in FIG. 12C, in an in-use position, the person's elbow (located at 616) is placed at the apex of the curved surface 612 and the arm slopes down one-half of the curved surface 612 as indicated.

Other accessories, such as tearable protective paper and one or more handles, can be provided.

Accordingly, FIGS. 12A-12E show that, in one or more embodiments, the body of the portable medical station has a curved top surface and a flat bottom so as to assume a semi-circular shape. In addition to one or more side drawers, the unit can include one or more openable lids that provide access to a hollow interior of the unit. In at least one embodiment, the openable lid can have a hinge at the apex of the curved top surface. A pad of tearable sheets can be provided also along the top surface and is intended for placement of the person's arm during the procedure.

Further, FIGS. 12A-12E show that, in certain embodiments, the drawers only open along one side. The arrangement of FIGS. 12A-12E can be used regardless of whether the health personnel is right-handed or left-handed, depending on the orientation of the unit 600. For example, to move between these two orientations, the unit 600 is simply rotated so as to position the drawers on the right side when a right-handed health professional is performing the phlebotomy and conversely, when a left-handed health professional is present, the unit is rotated such that the drawers are on the left side.

A bottom surface of any of the units described herein can have a non-slip grip surface, such as a rubber layer, etc.

Any number of different types of materials can be used to make the portable phlebotomy station including but not limited to plastics, wood, metal, etc. If plastics are used, much of the body can be formed as part of a molding operation which yields an integral structure. The drawers can be formed in part from synthetic textile materials attached to a drawer frame. Alternatively, the drawers can be formed of plastic.

Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. A portable phlebotomy station comprising:
a body having a sloped top surface on which an arm of a patient is for placement and a bottom surface with a hollow interior space being formed therebetween; and
one or more drawers that are disposed in the hollow interior space and movable between an open position and a closed position.

2. The portable phlebotomy station of claim 1, wherein the body has a wedge shape.

3. The portable phlebotomy station of claim 1, wherein the body includes a pivotable lid that defines the sloped top surface and provides access to at least one storage compartment defined thereunderneath.

4. The portable phlebotomy station of claim 1, wherein each drawer is accessible along at least one side of the body, each drawer being slidably received within the hollow interior space.

5. The portable phlebotomy station of claim 1, wherein the one or more drawers comprises a first drawer and a second drawer, the first and second drawers having different shapes.

6. The portable phlebotomy station of claim 5, wherein the first drawer has a trapezoidal shape and the second drawer has a triangular shape.

7. The portable phlebotomy station of claim 4, wherein each drawer is accessible and can be removed from the body along opposing sides of the body.

8. The portable phlebotomy station of claim 1, further including a handle attached to the body.

9. The portable phlebotomy station of claim 1, further including a pair of opposing straps attached to the body and configured to extend up and at least partially across the sloped top surface to permit fastening of free ends of the straps.

10. The portable phlebotomy station of claim 1, further including a pair of opposing straps attached to one drawer and configured to extend up and at least partially across the sloped top surface to permit fastening of free ends of the straps.

11. The portable phlebotomy station of claim 1, further includes a folder construction along a rear wall of the body for receiving objects.

12. The portable phlebotomy station of claim 1, further including a paper dispenser disposed along a rear wall of the body and configured with an opening to permit paper to be disposed up to a highest end of the sloped top surface.

13. The portable phlebotomy station of claim 1, further including a stack of sheets that can be removed one at a time and are disposed along the sloped top surface.
